(19) United States
(12) Patent Application Publication
(10) Pub. No.: US 2025/0360292 A1
(43) Pub. Date: Nov. 27, 2025

(54) SYSTEMS AND METHODS FOR INTERSTITIAL LIGHT DELIVERY

(71) Applicant: ABL Medical Inc., East Providence, RI (US)

(72) Inventors: Robert A. Rabiner, Dortmund (DE); Liane J. Rabiner, Dortmund (DE)

(73) Assignee: ABL Medical Inc., East Providence, RI (US)

(21) Appl. No.: 19/216,452

(22) Filed: May 22, 2025

Related U.S. Application Data
(60) Provisional application No. 63/650,680, filed on May 22, 2024.

(51) Int. Cl. A61M 25/06; A61M 25/00; A61M 25/01

(52) U.S. Cl. CPC A61M 25/0662; A61M 25/0102; A61M 2025/0056; A61M 2025/0681

(57) ABSTRACT

System, devices, and methods for providing treatment to tissue are disclosed herein. In some embodiments, a device is provided that includes a delivery catheter having an elongated shaft and an inner lumen therethrough, the delivery catheter being configured to pass through tissue such that a distal end of the delivery catheter is positioned at a target tissue, a support in the form of an inner slidable tube that is configured to be positioned inside the inner lumen of the delivery catheter, the support being configured to provide rigid or semi-rigid support for the delivery catheter during insertion of the delivery catheter through tissue, and one or more optical fibers configured to pass through the inner slidable tube and being configured to deliver light energy to provide an antimicrobial effect to the target tissue.

### RELATED APPLICATIONS

This application claims the benefit of and priority to U.S. Provisional Application No. 63/650,680 filed May 22, 2024, the contents of which is hereby incorporated herein by reference in its entirety.

### FIELD

The embodiments disclosed herein relate to treatments for tissue and/or bones, and more particularly, to antimicrobial blue light systems and methods for providing an antimicrobial, antibacterial effect for medical applications.

### BACKGROUND

Tissue and bone infection are critical issues in medical case. For example, bones form the skeleton of the body and allow the body to be supported against gravity and to move and function in the world. Bone fractures can occur, for example, from an outside force or from a controlled surgical cut (an osteotomy). A fracture's alignment is described as to whether the fracture fragments are displaced or in their normal anatomic position. In some instances, surgery may be required to re-align and stabilize the fractured bone. A bone infection may occur when bacteria or fungi invade the bone, such as when a bone is fractured or from bone fracture repair. These bacteria commonly appear and if not addressed properly can cause severe health problems. It would be desirable to have improved systems and methods for eliminating bacteria or other pathogens in tissues and bones.

### SUMMARY

The present disclosure is directed to system, devices, and methods for providing treatment to tissue. In some embodiments, a device is provided that includes a delivery catheter having an elongated shaft and an inner lumen therethrough, the delivery catheter being configured to pass through tissue such that a distal end of the delivery catheter is positioned at a target tissue, a support in the form of an inner slidable tube that is configured to be positioned inside the inner lumen of the delivery catheter, the support being configured to provide rigid or semi-rigid support for the delivery catheter during insertion of the delivery catheter through tissue, and one or more optical fibers configured to pass through the inner slidable tube and being configured to deliver light energy to provide an antimicrobial effect to the target tissue. The delivery catheter is configured to be movable between a first position in which the one or more optical fibers are positioned within the delivery catheter and a second position in which at least a distal potion of the one or more optical fibers are configured to extend past the distal end of the delivery catheter.

In some embodiments, a distal end of the delivery catheter includes a deflector component to divert a distal end at least one of the one or more optical fibers as it is being advanced from a distal end of the inner tube. In some embodiments, the deflector component includes a cut out portion and a distal ramp can that is configured to act as a deflector of the one or more optical fibers.

In some embodiments, the delivery catheter includes a proximal end with a head and a distal end having an angled tip.

In some embodiments, at least one of the delivery catheter or the inner tube includes one or more optical windows to allow light from the one or more optical fibers to escape from the delivery catheter or the inner tube. In some embodiments, locations and number of the one or more optical windows is based on the number of the one or more optical fibers or treatment required at the target tissue.

In some embodiments, the one or more optical fibers are configured to disperse the light energy evenly over a length of the one or more optical fibers in both longitudinal and circumferential directions. In some embodiments, the one or more optical fibers include a cladding covering an outer surface thereof, and wherein at least a portion of the cladding of the one or more optical fibers is removed from an outer surface of the one or more optical fibers to achieve the even dispersion of the light energy. In some embodiments, the antimicrobial effect of the light energy is configured to kill bacteria to treat infections.

In some embodiments, the light energy has illumination wavelengths from about 400 nm to about 475 nm. In some embodiments, the techniques described herein relate to a device, wherein the light energy has illumination wavelengths from about 380 nm to about 500 nm. In some embodiments, the techniques described herein relate to a device, wherein the light energy has illumination wavelengths from about 405 nm to about 470 nm.

In some embodiments, a device is provided that includes a delivery catheter having an elongated shaft and an inner lumen therethrough, the delivery catheter being configured to pass through tissue such that a distal end of the delivery catheter is positioned at a target tissue, the delivery catheter including a proximal head, a support in the form of an inner slidable tube that is configured to be positioned inside the inner lumen of the delivery catheter, the support being configured to provide rigid or semi-rigid support for the delivery catheter during insertion of the delivery catheter through tissue, and one or more optical fibers configured to pass through the inner slidable tube and being configured to deliver light energy to provide an antimicrobial effect to the target tissue. At least one of the delivery catheter or the inner tube includes one or more optical windows to allow light from the one or more optical fibers to escape from the delivery catheter or the inner tube.

In some embodiments, a distal end of the delivery catheter includes a deflector component to divert a distal end at least one of the one or more optical fibers as it is being advanced from a distal end of the inner tube. In some embodiments, the deflector component includes a cut out portion and a distal ramp can that is configured to act as a deflector of the one or more optical fibers.

In some embodiments, a method for treating tissue is provided that includes delivering a delivery catheter to a tissue such that a distal end of the catheter is positioned at a target tissue, delivering one or more optical fibers through the delivery catheter to the tissue, moving the delivery catheter between a first position in which the one or more optical fibers are positioned within the delivery catheter and a second position in which at least a distal potion of the one or more optical fibers extend past the distal end of the delivery catheter, activating a light source engaging the one or more optical fibers, and delivering light energy from the light source to the one or more optical fibers to provide an antimicrobial effect to the tissue, the one or more optical fibers dispersing the light energy evenly over a length of the one or more optical fibers in both longitudinal and circumferential directions.

In some embodiments, the antimicrobial effect of the light energy is configured to kill bacteria to treat infections. In some embodiments, the light energy has illumination wavelengths from about 400 nm to about 475 nm. In some embodiments, the light energy has illumination wavelengths from about 380 nm to about 500 nm. In some embodiments, the light energy has illumination wavelengths from about 405 nm to about 470 nm.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 illustrates an exemplary embodiment of a light fiber delivery device;

FIG. 2 illustrates an exemplary embodiment of an introducer having a proximal end with a head and a distal end having an angled tip;

FIG. 3 illustrates an exemplary embodiment of a light fiber delivery device with features to allow the location of the device to be tracked during insertion;

FIG. 4A, FIG. 4B, and FIG. 4C illustrate an exemplary embodiment of a method of inserting a light fiber delivery device to a target tissue;

FIG. 5A illustrates an exemplary embodiment of an optical fiber with a straight-line orientation that is positioned within the tissue site;

FIG. 5B illustrates an exemplary embodiment of an optical fiber having a curved or bent orientation;

FIG. 6 illustrates an exemplary embodiment of an inner slidable tube;

FIG. 7 illustrates an exemplary embodiment of the inner tube with one or more optical windows;

FIG. 8 illustrates an exemplary embodiment of the inner slidable tube with one or more optical windows;

FIG. 9A illustrates a side view of the introducer, or delivery catheter, having a cutout that can act as a diverter, ramp, and/or deflector;

FIG. 9B illustrates an embodiment of an introducer having a cutaway of the circular tube yet still maintains a means to keep the fiber contained;

FIG. 10 illustrates an exemplary graph showing fiber deflection;

FIG. 11, FIG. 12A, FIG. 12B, and FIG. 12C illustrates various exemplary embodiments of mechanisms to deflect a distal end of an optical fiber;

FIG. 13 is a perspective view of an exemplary embodiment of a steerable catheter;

FIG. 14 illustrates an exemplary embodiment of a graph showing a waveform of intensity of the specific frequencies;

FIG. 15A, FIG. 15B, and FIG. 15C illustrate exemplary embodiments of graphs showing that the frequency of the systems can remain the same while the power can increase, and the step up in power can result in faster and better kill of the target bacteria;

FIG. 15D and FIG. 15E illustrate exemplary graphs showing time versus bacteria reduction at different power settings;

FIG. 16A and FIG. 16B illustrate exemplary graphs showing how a plurality of LEDs can be used and can be dialed in via a light box at different powers;

FIG. 17 illustrates an exemplary graph showing power distribution over the length of an exemplary optical fiber;

FIG. 18A illustrates an exemplary embodiment of a fiber having cladding removed in a spiral pattern along the length of the light fiber;

FIG. 18B illustrates an exemplary embodiment of a light fiber having light emission from the tip of the fiber;

FIG. 19A and FIG. 19B show that an exemplary embodiment of a reflective surface added to the distal tip of a light fiber;

FIG. 20A, FIG. 20B, FIG. 20C, and FIG. 20D show various views of an exemplary embodiment of a fiber with cladding removed in a spiral;

FIG. 21 shows an exemplary embodiment of a fiber with a portion of the cladding removed;

FIG. 22 shows that in some embodiments, the cladding is removed 360 degrees around the fiber; and in some embodiments, the cladding is only removed in a 180 degree orientation with the cut side outwards; and

FIG. 23 illustrates an exemplary embodiment of a fiber having cladding that changes depth along the length of the fiber.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Systems and methods for antimicrobial blue light photolysis (ABLP) for treatment of tissue and/or bone infections and disorders are disclosed herein. In some embodiments, devices and methods for stabilizing and providing an antimicrobial effect for bone restructuring are disclosed. An antimicrobial effect may also include a bactericidal effect or an antibacterial effect, among other things. In some embodiments, the ABLP systems and methods can be used in conjunction with bone fracture fixation methods or other orthopedic procedures. For example, light for providing an antimicrobial and/or antibacterial effect can be used in a variety of medical applications, including but not limited to surgery, interventional radiology, respiratory and airway management, gynecology, dermatology, infectious diseases, wound care, and orthopedics.

Blue light has demonstrated antimicrobial properties against a range of microbes, including but not limited to gram-positive and gram-negative bacteria, mycobacteria, molds, yeasts, dermatophytes, and similar pathogens. In some embodiments, antimicrobial blue light having wavelengths between about 400 nm to about 470 nm can be used as an alternative to antibiotics.

In some embodiments, a device is provided for the percutaneous delivery of one or more light fibers for providing an antimicrobial effect and/or the treatment of tissue. For example, the light fibers can be used to treat small cancerous sites. For example, localized or site-specific areas of infection can be treated by the delivery of a light fiber to a specific location within the body.

In some embodiments, the one or more optical fibers can be delivered within a trocar, delivery catheter, or other insertion device. After insertion through tissue, the trocar, or delivery catheter, is withdrawn to expose the one or more optical fibers for the delivery of light within a tissue and/or organ. In some embodiments, a fluid, such as air, CO₂, water, or other fluid could be utilized as a distending member. Pressure from the fluid can be used to dissect tissue planes to open up and expose the area to be treated. The external body of the trocar, or delivery catheter, provides the strength and/or rigidity to allow for insertion of the device through the tissue. In some embodiments, the device includes the ability to deflect, move, and or manipulate the catheter and/or optical fibers to effect placement of the optical fibers in the body for treatment.

In some embodiments, delivery of fibers can be achieved via normal anatomic orifices, including nasal, oral, and urologic orifices. In some embodiments, there is a need to deliver fibers to locations in the body where no native orifice exists to treat any site-specific locations in the body.

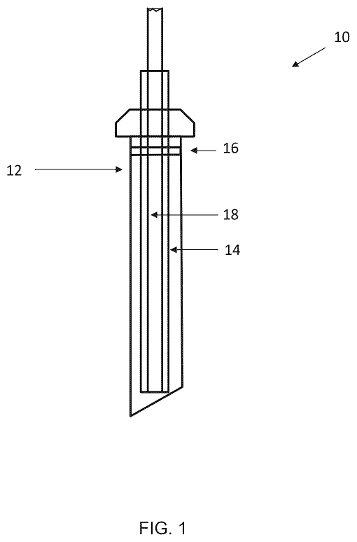

In some embodiments, a light delivery device can be provided that can be used to deliver one or more light fibers to a target tissue in the body in a minimally invasive manner. In some embodiments, delivery of fibers, such as small diameter fibers, can be achieved through the use of an introducer, for example, in the form of a trocar or a needle. In some embodiments, as shown in FIG. 1, the light fiber delivery device 10 can include an introducer 12 in the form of an elongate tube having a proximal end and a distal end with a lumen extending therethrough. For example, the introducer can be in the form of a trocar. A support, or inner slidable tube 14, can be positioned inside the lumen of the introducer 12 and can be configured to provide rigid or semi-rigid support for the introducer during insertion of the introducer through tissue. In some embodiments, the cap of the introducer has a seal 16 to prevent pressure loss, for example, if the procedure is being done during laparoscopic surgery when the abdomen or other body cavity is inflated with a gas, such as CO₂.

One or more light fibers 18 can be positioned inside the inner slidable tube 14 for delivery to a target tissue. The inner slidable tube 14 is designed to assist in the delivery of the one or more fibers, as well as to protect the one or more fibers from excessive bending and/or loads to prevent breakage. The fibers can be delivered to various target tissues, including but not limited to within the confines of the bone (e.g., to penetrate the periosteal surface of a bone to reach the intramedullary canal), within a site-specific tumor, nodules or cysts, abdominal or other cavities during laparoscopic surgery or other surgeries, and during arthroscopic procedures. The diameter of the slidable tube/inner lumen of the introducer can vary depending on the size and number of fibers being introduced therethrough.

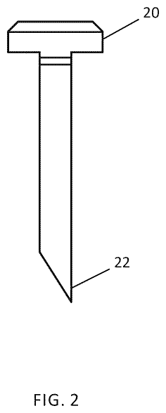

The introducer can include various features to assist in penetrating the tissue to deliver the one or more fibers. FIG. 2 illustrates an exemplary embodiment of an introducer having a proximal end with a head 20 and a distal end having an angled tip 22. The angled tip 22 at the distal end of the introducer is configured to pierce through tissue to position the distal end of the introducer adjacent the target tissue to be treated. The sharp distal tip is configured to penetrate skin and/or facia for passage through the subcutaneous material towards the target tissue for the delivery of the one or more light fibers.

To prevent damage to the fiber and to prevent damage to tissue by the sharp tip of the needle, the fiber can be delivered via the inner slidable tube within the outer introducer wall. In some embodiments, the inner slidable tube can be constructed from metal or polymer.

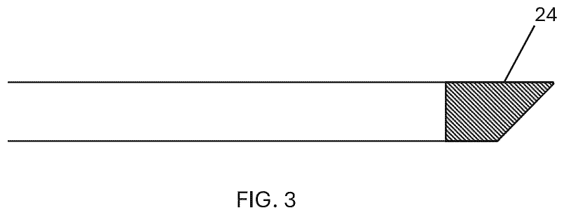

In some embodiments, the tip of the tube can include features to allow the location of the tube to be tracked during insertion, as shown in FIG. 3. In some embodiments, the tip of the tube has a surface finish such that it is reflective to ultrasound so that it can be guided to a specific location using ultrasound during insertion. In some embodiments, the tip of the tube can have a radiopaque marker band 24 on it to locate and identify the position of the tube by fluoroscopy. In some embodiments, the tube can include multiple radiopaque markers thereon, for example, to track depth of the tube through the tissue during light fiber delivery.

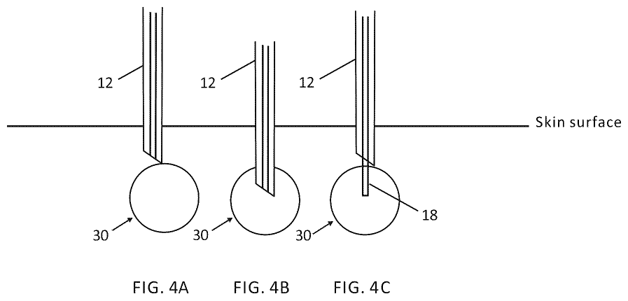

As shown in FIG. 4A, FIG. 4B, and FIG. 4C, the introducer can be configured to penetrate the skin and move through tissue to position the distal tip of the introducer within the tissue of concern 30, such as a tumor or a focal point of infection. After delivery of the introducer to the target tissue, the introducer can be move in a proximal direction and/or withdrawn to expose the one or more optical fibers to deliver light energy to the tissue. As shown in FIG. 4A, the fiber is contained within the tube of the sharp introducer 12 (e.g., a larger lumen needle). The slidable inner tube provides strength and stability for the introduction of the one or more fibers 18. FIG. 4B shows the sharp needle being driven into position. As shown, the introducer 12 has pushed through the skin surface and to a target tissue 30. In some embodiments, the tip of the needle is radio opaque and can be surfaced so as to provide better acoustic properties so that ultrasound/sonography can visualize the tip position. The tip is driven into the correct position in the target tissue, or tissue of concern, 30. As shown in FIG. 4C, the sharp tip of the introducer 12 is withdrawn from the target tissue 30 and the one or more light fibers 18 remain in position relative to the target tissue 30, leaving the one or more light fibers 18 to be illuminated in the correct position within the tissue.

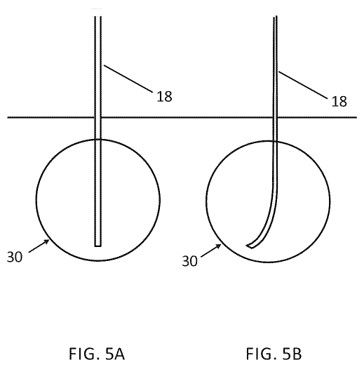

It is also possible for the light fibers delivered to the tissue to have various confirmations. For example, a portion of the light fibers or the entire length of the light fibers can be straight or bent. As shown in FIG. 5A, in some embodiments, a fiber with a straight-line orientation is positioned within the tissue site. As shown in FIG. 5B, in some embodiments, a fiber can have a curved or bent orientation. It is possible for the curved or bent orientation of the light fibers to be achieved in a variety of ways. In some embodiments, the fiber can be positioned in the tissue and deflected. For example, a distal portion of the one or more of the light fibers can be deflected once the light fibers are positioned at the target tissue to effect specific locations within the target tissue. In some embodiments, the fiber can be pre-bent but held in a substantially straight orientation when positioned within the lumen of the introducer. Thus, the pre-bent portion of the light fibers can bend when the introducer is moved proximally to expose the light fibers positioned at the target tissue. In some embodiments, the fiber can have a straight orientation and can be deflected by the shape of the tip of the catheter/introducer as the catheter/introducer is moved proximally and retracted from the tissue to leave the fiber behind in the target tissue.

In some embodiments, the proximal end of the introducer can include a sealing member, as shown in FIG. 1. The sealing member can have a variety of purposes. In some embodiments, the sealing member can be configured to hold the inner slidable tube in position relative to the introducer. In some embodiments, the sealing member can be configured to seal the introducer relative to the environment. For example, should the location that the needle is place in be pressurized, for example, during use in a laparoscopic procedure, the sealing member can prevent egress of pressure from the body cavity. In some embodiments, the inner slidable tube can also include a sealing member positioned at the proximal end thereof and can be used to seal the fiber delivery port. In some embodiments, the seal can be used to control the environment for the procedure while allowing fluid, such as air or saline, to be infused into the treatment site as needed. For example, the fluid can be used to increase light transmission of the light from the one or more optical fibers through the fluid media.

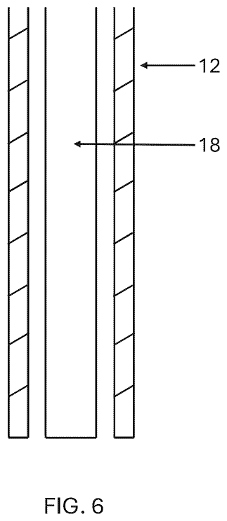

The shape of the inner slidable tube, as shown in FIG. 6, can vary depending on the location of the body being accessed by the tube. In some embodiments, the inner slidable tube can be straight. In some embodiments, the tube can be angled or bent before insertion, but can straighten as it is withdrawn into the outer needle/trocar. The inner slidable tube can be advanced forward and backwards and can be rotatable as needed to assist in positioning of the one or more optical fibers. For example, when the inner tube is prebent or shaped, this can permit the steering and/or deflection of the light fiber.

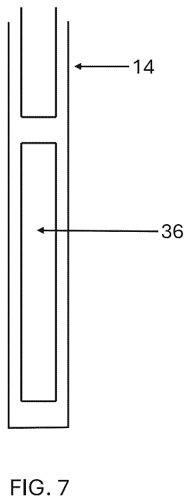

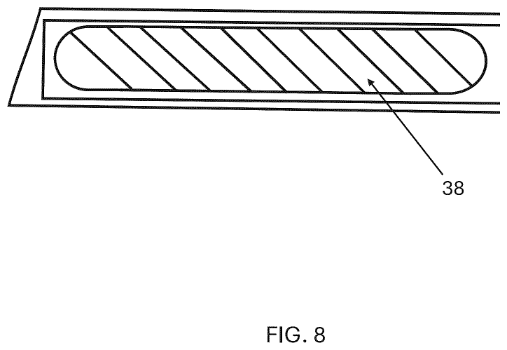

The inner tube is also configured such that light energy from the one or more optical fibers positioned therein can be transmitted to the target tissue or bone. In some embodiments, the inner tube can be formed from a clear material that allows the transmission of light. In some embodiments, the inner tube can include one or more optical windows 36, as shown in FIG. 7, to allow light to escape from the introducer and/or the inner tube. For example, the locations and number of the optical windows can depend on the type and number of fibers in the inner tube and/or the treatment required at the target tissue.

As explained above, in some embodiments, as shown in FIG. 8, the inner slidable tube (i.e., the support tube for the one or more fibers) can include one or more optical windows 38. This allows the inner slidable tube to support the one or more fibers during delivery and placement of the one or more fibers into the target tissue while also allowing the optical energy to be delivered. In some embodiments, the inner slidable tube can be metal with one or more windows. In some embodiments, the inner slidable tube can be formed from a clear polymer tube allowing transmission through the tube. In some embodiments, the inner slidable tube can be formed from a clear polymer with one or more windows formed therein to alleviate transmission loss through the polymer.

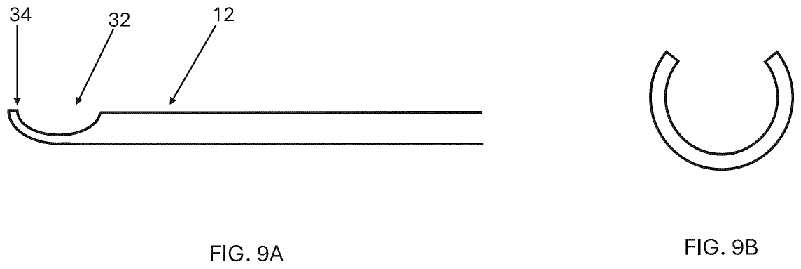

In some embodiments, the introducer can include a deflector component or a diverter, for example, in the form of a cutout portion. For example, the cutout portion at the distal end of the introducer can be configured to shape and/or deflect and/or divert a distal portion of the one or more optical fibers. FIG. 9A illustrates an exemplary embodiment of a side view of the introducer/catheter having a cutout at the distal end that can act as a diverter/ramp/deflector. The design can act to divert or deflect the distal end of the one or more fibers as the fibers are being advanced from the tube of the introducer/catheter as the tube is moved proximally, while being able to contain the fiber using the “c” shape of the tube formed by the cut out portion. This forces the fiber to be deflected outwards. The cutaway or cut out portion 32 of the tube plus the ramp 34 can act as a deflector of the fiber.

FIG. 9B illustrates an exemplary embodiment of an introducer having a cutaway of the circular tube yet still maintaining a means to keep the fiber contained.

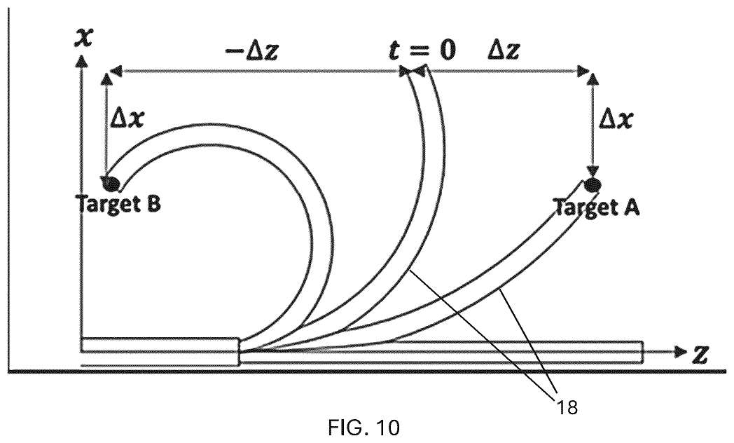

FIGS. 10, 11, 12A, 12B, and 12C illustrate exemplary embodiment of optical fibers with at least a distal portion thereof being deflected, curved, or bent. For example, FIG. 10 illustrates an embodiment of a plurality of optical fibers 18 that are prebent before use. The structure of the introducer and/or the inner tube allow the prebent fibers to be inserted into the body in a generally straight configuration, and they can then expand into the prebent configuration after deployment from the introducer. For example, after the introducer is moved proximally to expose at least a portion of the distal ends of the fibers, the fibers are able to expand into their prebent configuration. The orientation of the distal end of each fiber can be such so as to position the distal end of each fiber in a desired position relative to the target tissue. Thus, each optical fiber can have a different deflected position or have different angle of deflection if needed.

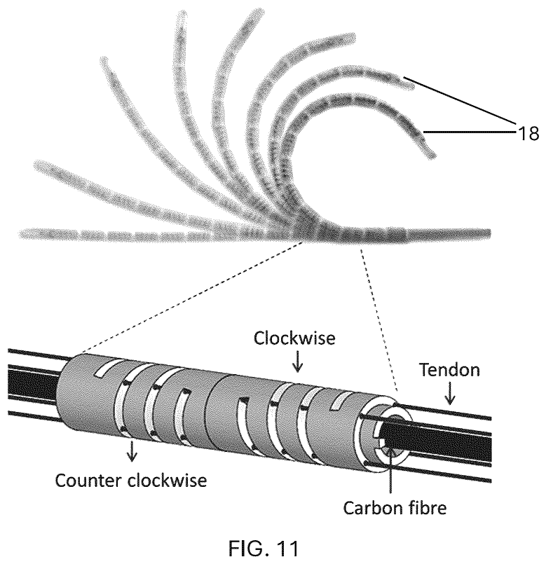

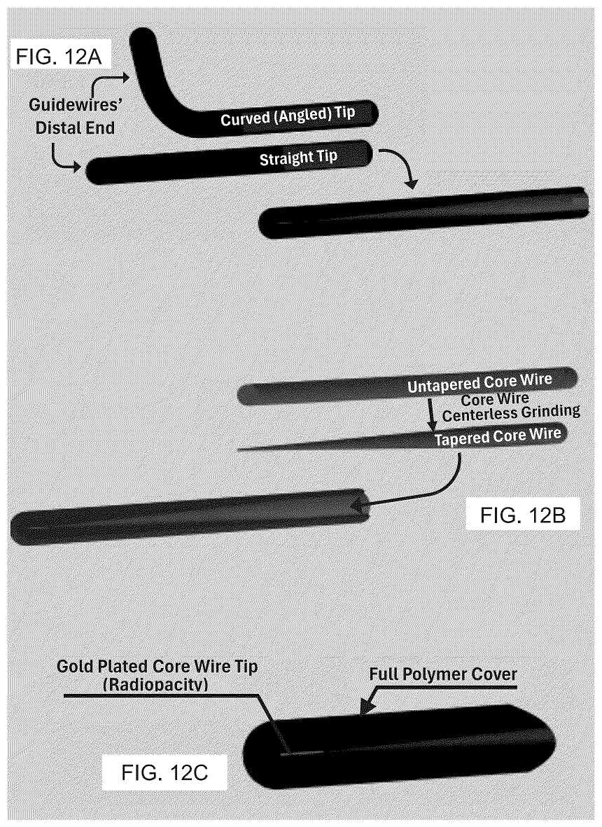

In some embodiments, the one or more optical fibers can be deflected or bent after insertion into the body using a controller to adjust the deflection of the fibers. FIG. 11 illustrates an exemplary embodiment of a controller mechanism that can push and/or pull on a catheter with embedded wires to adjust each optical fiber 18 to a desired position relative to the target tissue.

In some embodiments, a guidewire, spiraled component, or other device can be used to deliver the fibers. The guidewire can be prebent to deliver the optical fibers in a deflected manner, or the guidewire can be relative straight to deliver prebent optical fibers, as shown in FIG. 12A, FIG. 12B, and FIG. 12C.

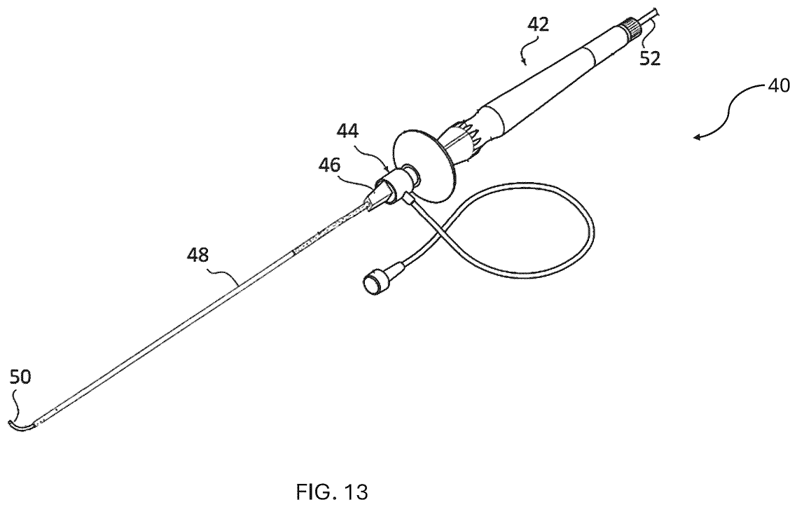

In some embodiments, a steerable catheter can be used to deliver the optical fiber to the target tissue. Referring to FIG. 13, an embodiment of a steerable catheter 40 is shown and includes a handle means 42 connected to a fitting 44 which is in turn is connected to an attaching means 46 fixed to an outer sheath 48. A catheter tip 50, which is connected to a catheter hereinafter described, extends from the outer end of the sheath, and a medical device 52, such as an endoscope, extends through the handle means 42 and is supported within the catheter.

The handle means 42 supports an elongated outer sheath and an elongated catheter extends through the sheath. The catheter is movable lengthwise inside of the sheath and is rotatable with respect to the sheath. The outer end of the catheter comprises a memory tip which causes the tip to be disposed at a desired angle to the sheath when the tip is extended a certain distance from the sheath.

The optical fiber used in the device can be made from any material, such as glass, silicon, silica glass, quartz, sapphire, plastic, combinations of materials, or any other material, and may have any diameter. Further, the optical fiber can be made from a polymethyl methacrylate core with a transparent polymer cladding. It should be noted that the term “optical fiber” is not intended to be limited to a single optical fiber but may also refer to multiple optical fibers as well as other means for communicating light from the light source to the expandable member. It is possible the fibers, after exciting the light source, may be twisted so as to form into a single fiber. Further, the optical fiber may comprise of a single fiber at a location that is in combination with multiple fibers at another location. It is possible that the multiple fibers positioned at the other location may be further incorporated into another single fiber at yet another location within the system, i.e., the method of using the light fiber may be a single fiber or multiple fibers or any variation thereof.

If a prescribed dose (for example, intensity or some other measurement associated with the light) is defined as the means to achieve an antimicrobial effect, then in some embodiments, that dose/amount of energy needs to be delivered over the entire active length of the fiber for the affected area to be treated. For example, the light emission of the fiber can be in a helical coil around an active area of the fiber to provide a larger area of treatment.

In some embodiments, for example, in the case of a target site being a single location where illumination can be directed, similar to the effect of a flashlight or spotlight, the light emission of the fiber can be at the distal tip of the fiber for site specific delivery.

The size of the one of more fibers can vary. For example, the light fibers can have a diameter of 0.5 mm, 0.75 mm, 1.0 mm, 1.5 mm, or 2.0 mm. The fiber having a small diameter (e.g., ˜3 mm or smaller, ˜3 mm OD-1.75 mm OD) can be used to penetrate the skin to reach subcutaneous locations. It will be understood that any size fiber can be used depending on the needs of the target tissue, and that the inner diameter of the introducer and the inner tube will be sufficient to receive the one or more fibers.

In some embodiments, the light source includes a single frequency or plurality of frequencies of the light energy. In some embodiments, the plurality of frequencies of the light energy are selected based on the antimicrobial effect on specific microbial targets for each of the plurality of frequencies of light energy. In some embodiments, a subset of the plurality of frequencies of light energy can be used based on the specific microbial targets. In some embodiments, the light energy has illumination wavelengths from about 400 nm to about 475 nm.

In some embodiments, the light source emits frequency that corresponds to a band in the vicinity of 350 nm to 770 nm, the visible spectrum. In some embodiments, the light source emits frequency that corresponds to a band in the vicinity of 380 nm to 500 nm. In some embodiments, the light source emits frequency that corresponds to a band in the vicinity of 430 nm to 450 nm. In some embodiments, the light source emits frequency that corresponds to a band in the vicinity of 430 nm to 440 nm.

For example, in some embodiments, the blue light/beam can have a wavelength of about 405 nm, about 420 nm, about 450 mm, about 460 nm, or about 470 nm, or any other wavelength that can damage various bacteria. In some embodiments, multiple single spectrums can be used, e.g., 405 nm and 420 nm. The individual frequencies of light can be mixed/focused to provide two or more frequencies within a single fiber. For example, in some embodiments, the blue light/beam can have a wavelength of about 405 nm, about 420 nm, about 450 nm, or about 470 nm.

The other rationale for the use of multiple LEDs is selecting frequencies that are known to have an antimicrobial effect on the specific microbial target. For example, some bacteria can be remediated with specific frequencies, while other bacteria are not affected, or are affected at lower levels. Through the ability of merging multiple light frequencies, the user can either pick the appropriate light for the bacteria or can apply multiple frequencies to remediate the bacteria.

It is contemplated that the light source can include a single bulb or multiple bulbs. For purpose of clarity, “bulb” is used as an indiscriminate description of a light source. A bulb may be a metal halide source, a mercury or xenon incandescent, or LED. The type of light source can vary, and can be in the form of one or more LEDs, a laser, or any other potential light source that can provide the desired wavelength of light. The light source may further include one or multiple ports to attach light fibers. The light fibers or light guides may be joined, mixed or include some combination thereof, within the system. Depending upon the application, the light source can be designed to provide higher outputs in different frequencies, i.e., using multiple bulbs, so as to overcome potential fall-off aspects that may occur using a single bulb. If multiple bulbs are used, it is contemplated that there may be multiple types of bulbs used in the system. For example, each different type of bulb may provide a specific attribute to meet an intended design aspect for the particular application, which may include attributes relating to frequency ranges, energy density ranges, operation life expectancies, etc. Further, regarding other elements within the system where multiple elements of the same element are used, i.e., light fibers (optical fibers, light guides, etc.), light conductive materials and the like, it is contemplated that there may be different types of the same element used within the system. As noted above, each different type of element may be used depending upon the specific attribute to meet an intended design aspect for the particular application, which may include attributes relating to material type(s), performance related ranges, operation life expectancies, etc. In conjunction with choosing a specific element, any materials and elements used with that specific element may be further used, so as to meet the intended planned design for the particular application. For example, it is contemplated a clear liquid epoxy may be used to bind and fill in interstices of multiple fibers towards a smooth tube or the like, with the system.

In some embodiments, there can be multiple light sources coupled to different components of the system. For example, a first light source can be coupled to the proximal end of one or more fibers, and a second light source can be coupled to the distal end of the one or more fibers.

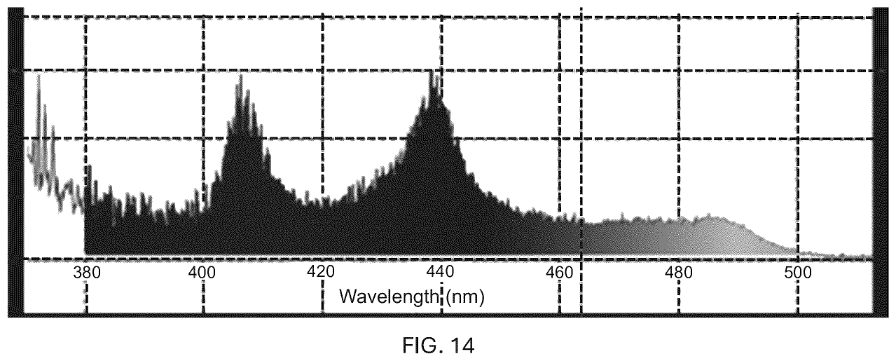

In some embodiments, a metal halyide bulb can be used. As shown in the exemplary graph in FIG. 14, the waveform provides “peaks and valleys” such that specific spectrums are naturally higher than others as a function of the bulb/light that is illuminated from the bulb, but the intensity of the specific frequencies within the waveform cannot be changed. Looking at the power of the various frequency bands as a percentage of the total power delivered, it is shown that most of the frequencies outside the 400 nm-500 nm range are fairly low in power percentagewise.

If a specific frequency/intensity is needed to affect the kill of the bacteria, and that intensity is lower than needed, there is no means to increase the power without raising all the other frequency powers. This runs the risk of potentially inducing more power than is required and at the risk of potential damage to normal cell viability.

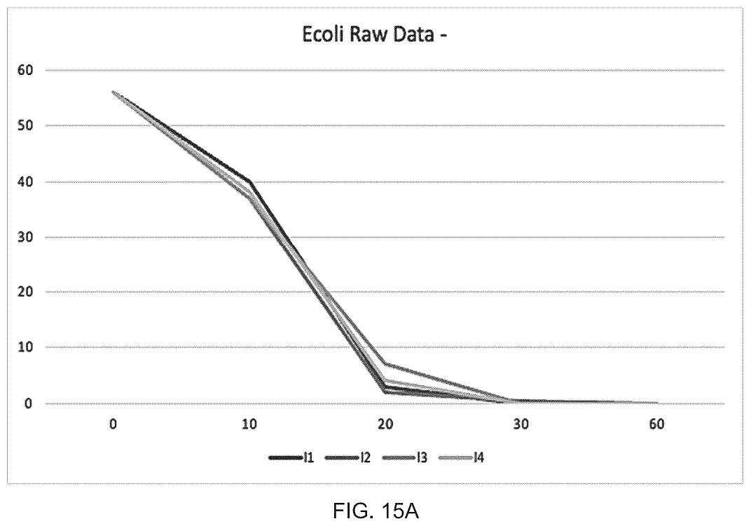

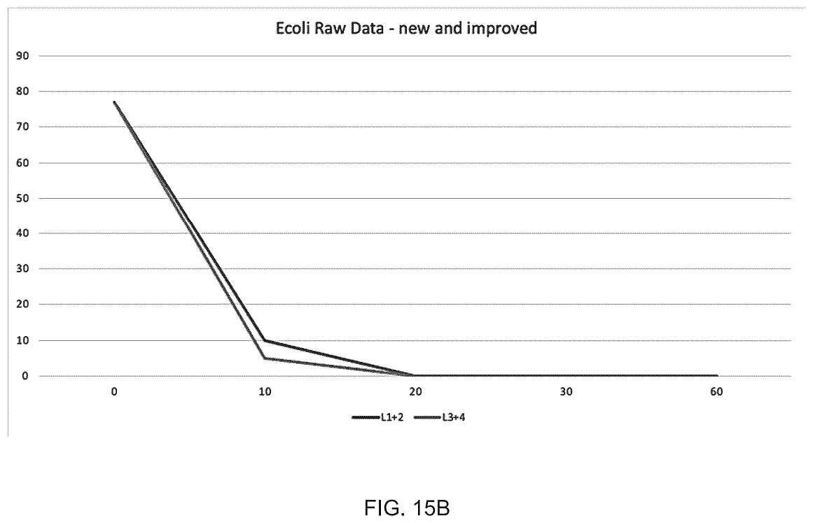

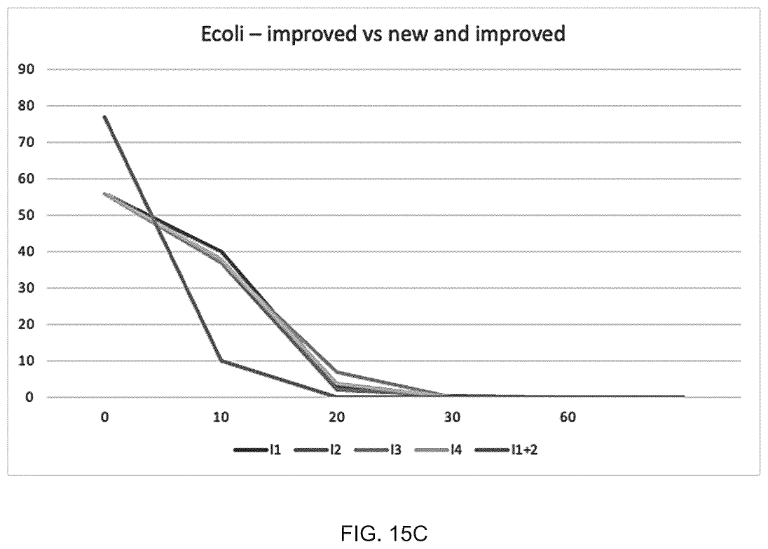

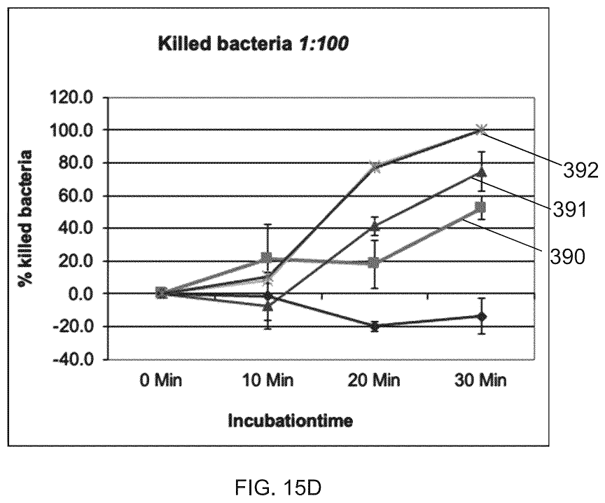

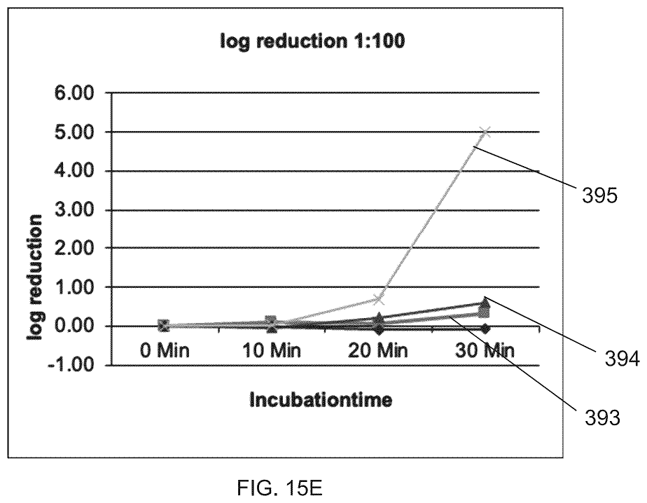

As shown in FIG. 15A, FIG. 15B, and FIG. 15C, the frequency of the systems can remain the same while the power can increase, and the step up in power can result in a faster and better kill of the target bacteria. Thus, more power results in a more effective bacteria elimination. Similarly in FIG. 15D and FIG. 15E, which illustrate exemplary graphs showing time versus bacteria reduction at different power settings, high power correlates to an increase in bacteria reduction. Three different power levels are shown in FIG. 15D, with lines 390, 391, 392 going from lowest to highest power. Similarly, three different power levels are shown in FIG. 15E, with lines 393, 394, 395 going from lowest to highest power.

A plurality of specific LEDs at the specific frequencies can be used at frequencies that are desired or needed to cause an antimicrobial effect. This allows the frequency and the intensity delivered to the tissue to be more defined and specific as the intensity of each LED is controlled-the delivered intensity at the various frequencies can be the same if desired, versus the peaks and valleys of the metal haylide.

If specific frequencies of light are appropriate in the remediation of one bacteria, while other frequencies are not, those frequencies can be turned off as there is no reason to deliver light to the treatment zone if it is not beneficial. Thus, it is possible to provide the desired frequencies of light using a subset of the plurality of LEDs as needed for each specific bacteria. This can provide a variable and tunable system.

The “tuning” of the system is relegated to the application or use of specific LEDs as each LED is a single frequency light. Thus, the system does not have the ability to adjust the LEDs other than adjusting the power intensity up or down. The system uses the combination of the available LEDs to create an appropriate blend of frequencies to achieve a blend of light frequencies to achieve the appropriate kill factor for a given bacteria. The system can adjust the power to increase or decrease the peaks (i.e., power intensity) of the various frequencies. In some embodiments, this can be done to ostensibly derive a square wave to create a block of power hitting the bacteria.

It will be understood that frequency and/or power are responsible for the killing of bacteria. In some embodiments, the frequency and/or power of the plurality of LEDs can be tuned in an attempt to target specific bacteria. In some embodiments, a more broad-spectrum approach can be used with the plurality of LEDs. For example, using high power at (seemingly) the wrong frequency can provide a null response. In addition, as many physicians do not know the specific strain of bacteria that is affecting a patient, the effects of using a broad-spectrum light system can outweigh negatives of frequency specificity in an attempt to target a specific bacteria.

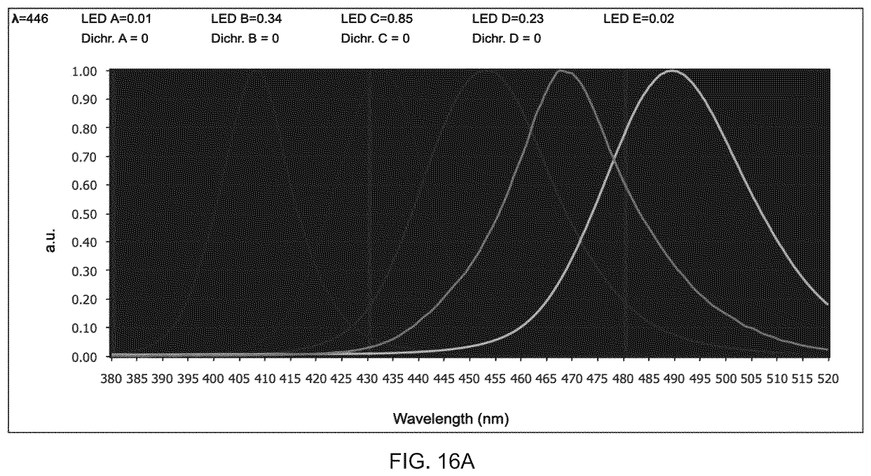

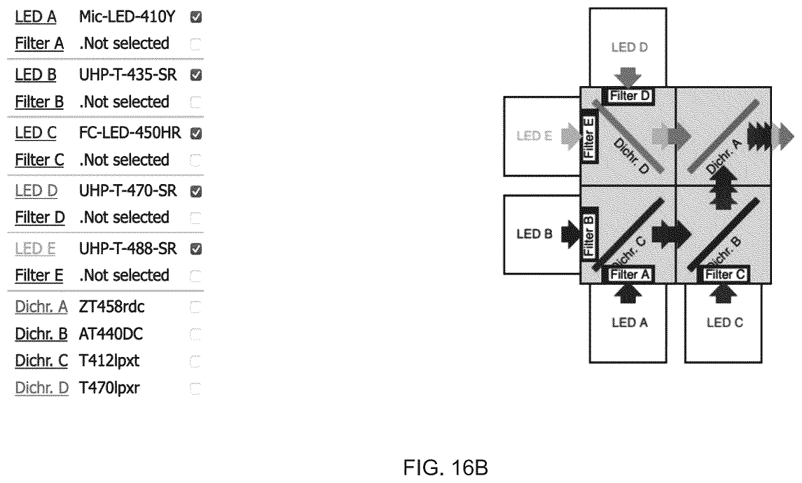

In some embodiments, a plurality of LEDs, as shown in the exemplary graphs in FIG. 16A and FIG. 16B, can be used and can be dialed in via a light box (i.e., switches thrown to pull specific frequencies), and the ability to drive the various LEDs at different powers (so that the optical output is the same). This allows for “ganging up” of multiple LEDs with different frequencies, and optical mirrors can be used to merge the multiple light frequencies into a single plastic optical fiber. Different LED frequencies can have different optical output, e.g., intensity. The output can be adjusted through the drive current, where overdriving them will result in higher optical output. The multiple LEDs ganged up can be used to fine tune the frequency/frequencies of light that are delivered to the fiber in the treatment of the bacteria, as certain species of bacteria have different frequencies of light that are able to kill them. This allows the system to target the light to the species of bacteria to make it a more targeted system.

The light fiber can be used alone, utilizing the body's natural response to blue light without photosensitizers, or in combination with photosensitizers. Photosensitizers can be used with photodynamic therapy (PDT) to treat malignant diseases using the photosensitizer, a light source, and oxygen. The combination initiates a photochemical reaction that generates singlet oxygen that produces a local cytotoxic effect to destroy unwanted cells or tissues. Using PDT, an optical fiber can be placed directly or immediately adjacent to a lesion to target the destruction of tumor cells while protecting the surrounding tissue.

Multiple fibers can be used (e.g., multiple light boxes or multiple attachment points on the light box permit the use of more than one fiber).

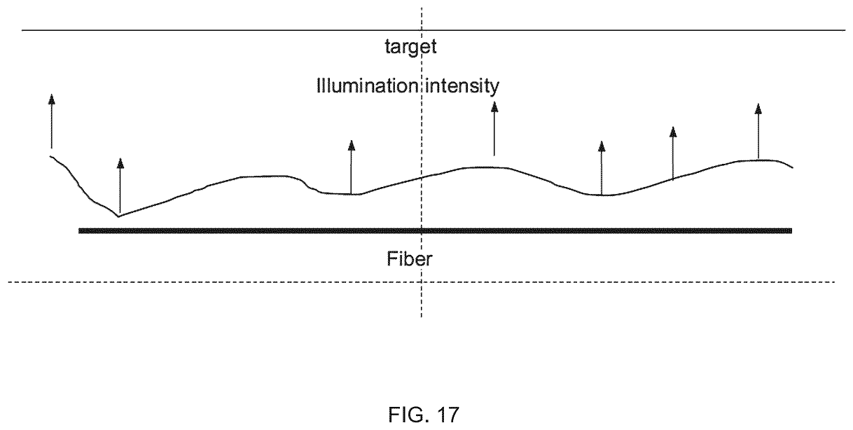

The light delivered by the fiber needs to be even over the length of the fiber such that the correct power over the length of the fiber can be provided, at even powers at short lengths over the fiber to achieve the antimicrobial effect, as shown in FIG. 17. If there is an imbalance in the power over the length of the fiber, there can be high spots and/or low spots, and the patient suffers as there isn't any way to achieve a balance in treatment. If there are high spots and/or low spots (areas of increased intensity and/or areas of decreased intensity), then there is not even power deposition to the targeted area needing treatment. This would correlate to overmedication or undermedication of the treatment site. Thus, the correct power can be selected for an even power distribution over the length of the fiber.

In some embodiments, uniform light delivery along the length of a fiber can be achieved by virtue of a variable helical spiral. Removal of cladding in this manner allows the system to maintain the same energy deposition over the length of the fiber such that the top and the bottom are even and light emanating from all planes of the fiber are uniform. This allows for 360 degrees of light over the length of a fiber.

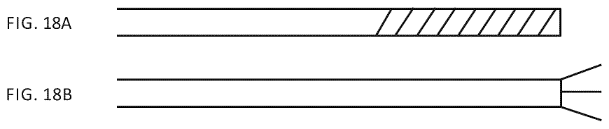

For example, as shown in FIG. 18A, cladding can be removed in a spiral pattern along the length of the light fiber. This creates a side emitting optical fiber, the isotropic fiber. This is in contrast to an embodiment, shown in FIG. 18B, where a light fiber only has light emission from the tip of the fiber (e.g., a “flashlight”).

The type of light fiber can vary, and can include traditional fiber optics, telecommunication fiber, or plastic fiber optics that can be efficient in the transmission of light with minimal light loss. It should be noted that with any form of diffusing/diffusion light fiber, the intensity of the light will decrease over length of the fiber dependent upon the amount of light being diffused (length and/or area).

A process of even diffusion of the light in the cladding over the length of the fiber results in stronger intensity at the initiation end of the fiber and an ever-decreasing amount as distance is increased from the initiation source. This reduction in optical power and intensity negates its use in the curing of photodynamic implants, as the intensity at the distal end has weakened significantly (or the increased power to achieve curing at the distal end of the fiber has been increased so significantly that there is an overpowering of the fiber at the proximal end). Thus, a variable helix of a cut in the cladding, spiraling down the fiber, with the spiral getting tighter and tighter as the light is bleeding out, allows for even light dispersion over the length of the fiber.

In some embodiments, an antimicrobial system can include an optical fiber having a diameter in the range of 1 mm to 20 mm, with a light emitting helical coil on the circumference of the fiber. The illumination of the fiber is delivered radially from the fiber outwards. Illumination frequencies are in the visible spectrum from about 400 nm to about 475 nm.

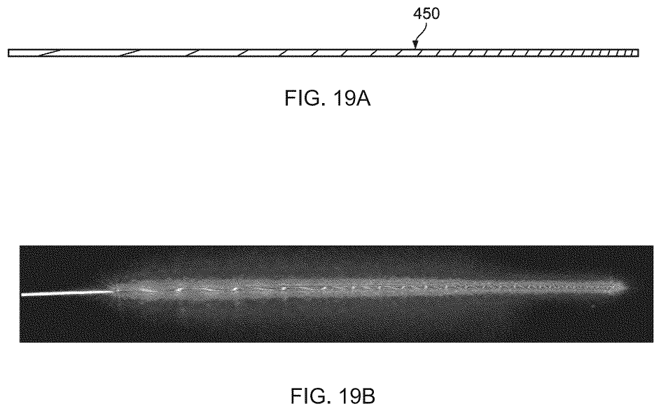

Thus, in some embodiments, a fiber 450 can have a spiral/helical coil of cladding that can be removed, allowing light to escape from within the fiber to affect the ABLP process, as shown in FIG. 19A and FIG. 19B. The cladding is removed such that the light intensity along the length of the fiber is uniform. Traditionally, PMMA (acrylic) comprises the core (96% of the cross section in a fiber 1 mm in diameter), and fluorinated polymers are the cladding material. Since the late 1990s much higher performance graded-index (GI-POF) fiber based on amorphous fluoropolymer (poly (perfluoro-butenylvinyl ether), CYTOP) has begun to appear in the marketplace. Polymer optical fibers are typically manufactured using extrusion, in contrast to the method of pulling used for glass fibers.

In some embodiments, cladding is removed physically (i.e., scratching the surface in a very precise method using, for example, a diamond tipped cutting head, a razor, or scalpel blade) which can reveal the fiber and allow light to emanate through the space in the cladding. In some embodiments, cladding is removed chemically from a polymer optical fiber using organic solvents which can also be used to create etched portions of the fiber allowing the attenuation of light. Cladding can also be removed using low energy lasers to finely ablate the surface, water jet cutting, or with compressive dyes to penetrate/break the surface of the cladding.

To provide for uniform light delivery, when the cladding is removed along the length of the fiber, the spiral can tighten as it progresses from a proximal end of the fiber to a distal end of the fiber. In some embodiments, the depth of the cut into the fiber can also increase from the proximal end of the fiber to the distal end of the fiber.

The greater penetration depth of the fiber towards the dispersion of light requires increased penetration depth as the light intensity also decreases through the attenuation or loss of light though the removed cladding area.

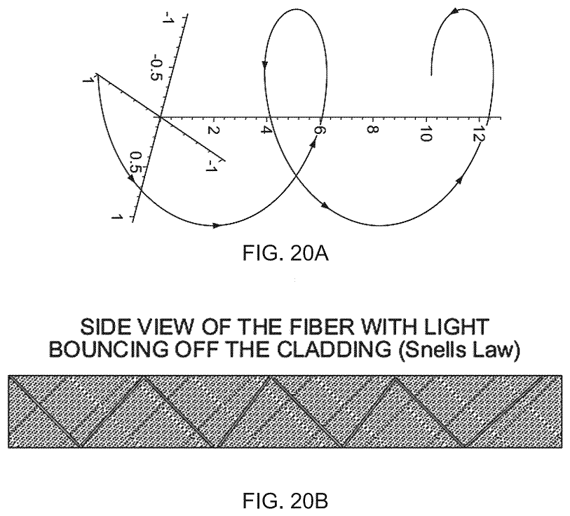

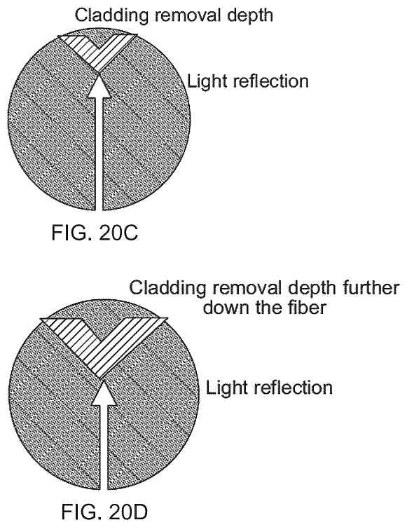

For example, a spiral design that wraps around the fiber on the line of the spiral, where the cladding has been removed to allow light to escape, can be used, as shown in FIG. 20A, FIG. 20B, FIG. 20C, and FIG. 20D. As the light escapes, the intensity of the light being emitted is reduced from 100% of the light at a proximal end of the fiber closest to a light source, and as the light moves distally further and further down the spiral, there is less light coming out of the fiber (the light intensity decreases). In order to resolve the issue, the pitch can be increased to narrow the gap between the two spirals and to increase the amount of light hitting the target. However, as the light moves further and further down the fiber and the intensity is dropping, the depth of the cut in the cladding can be increased to decrease the distance that the light within the fiber needs to transit before exiting the fiber. Thus, in order to achieve the necessary amount of light along the entire fiber length, there is a balance of the spiral and the depth of the cladding cuts to achieve an even light distribution over the length of the fiber. For example, an initial cut in the fiber can be shallow such that only the cladding is removed. The cuts can become deeper distally along the length of the fiber (i.e., thousandths of an inch deeper).

In some embodiments, the cut in the cladding can also vary in depth, with the proximal aspect of the fiber cut only a minimal depth (for example, ˜10 micron depth) to allow the light to pass through to an increasing depth of cut as the spiral moves down the length of the fiber. This is illustrated in plots based on pitch (mm), cutter rpm and cutter amps, with the increased amps yielding a deeper cut.

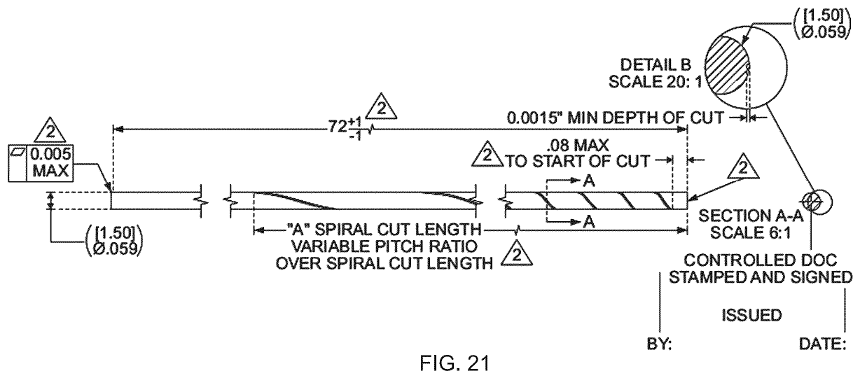

In some embodiments, the removal process includes holding the fiber in a rotary chuck with the cutting point mounted on a motor. A spring position can be used to advance the pointer and make contact with the cladding, and the spring drive holds the pointer in contact with the fiber. While the increased pitch and cutter rpms are applied, the cutter system is rotated around the fiber to apply the spiral cut (as does the increased depth of the cut), as shown in FIG. 21. As shown in FIG. 21, there is an increasing helical pitch, including a slow/shallow pitch at the proximal end and a very tight pitch at the distal end. It will be understood that the pitch becomes tighter from the proximal end to the distal end of the fiber. Further, the depth of the cut increases as the cut progresses down the length of the fiber from the proximal end to the distal end.

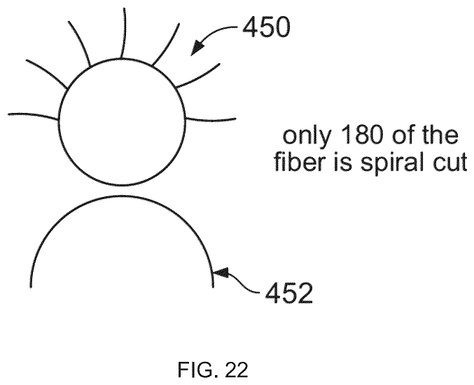

In some embodiments, the cladding is removed from only certain portions of the fiber. In some embodiments, the cladding is removed 360 degrees around the fiber. In some embodiments, the cladding is only removed in a 180 degree orientation, as shown in FIG. 22, with the cut side outwards. As shown in FIG. 22, the uncut side of the fiber 450 can be positioned against a balloon 452. The 180 degree cut side is exposed to the endosteal surface. By having the spiral cut only on 180 degrees of the fiber, the intensity of the light increases. The reduction in the cut cladding can increase the output efficiency. In some embodiments, the cladding is removed in a 90 (45+/−) degree orientation, with the 90 degree cut side exposed to the endosteal surface. This reduction in the cut cladding can also increase the output efficiency. In some embodiments, the cladding is removed in a 120 (60+/−) degree orientation, with the 120 degree cut side exposed to the endosteal surface. The reduction in the cut cladding can increase the output efficiency. It will be understood that any amount of the circumference of the cladding can be removed from the fiber to control the amount of light from the fiber and the area of tissue being exposed thereto. The cladding can be removed in the direction of intended light delivery.

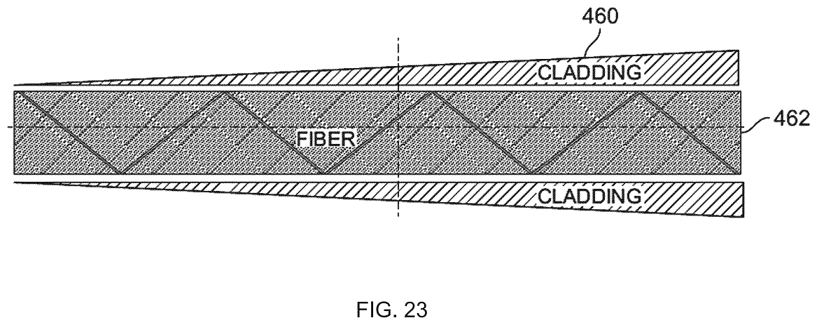

Based on cladding removal, as shown in FIG. 23, energy dissipation remains the same, or substantially the same, as length increases. Cladding cut width is the same, but the depth of cut increases over the length so light emanates stronger toward the distal end. A helical spiral can be cut with a slower pitch at the proximal end, and the pitch can increase as it moves toward the distal end, creating a tighter spiral.

In some embodiments, rather than removing a portion of the cladding from the fiber to achieve uniform light energy delivery/power deposition along the length of the fiber, one or more fibers without cladding can be used to deliver the light energy uniformly. When using a fiber without cladding, the fiber can be overcoated with a light diffusing membrane. The over coating can be applied (or an extrusion) where the leaking of light through the diffusion membrane is low at the proximal end of the fiber (or light intensity side) and the diffusion can gradually increase as the fiber gets longer towards the distal end of the fiber. The outer membrane can be scaled to allow for uniform/even light and power deposition along the length of the fiber.

A diffusive membrane can be deposited in specific thicknesses or patterns on the fiber to achieve uniform delivery of light. In some embodiments, segments of diffusive material can be applied to the outer surface of the fiber in an arrangement that provides uniform light along the length thereof. In some embodiments, a diffusive coating (e.g., a spray coat, dip coat, or ionic deposition coating) can be applied to the fiber such that the thickness of the coating decreases along the length from the proximal end to the distal end of the fiber. The thinning of the coating towards the distal end allows for an increase in the amount of light diffusion through the coating from the proximal to the distal end such that the end result is uniform delivery of light along the length of the fiber. As described herein throughout the disclosure and the various embodiments, the energy across the length of the fibers (the linear deposition of even power) is uniform such that the bacteria or other microbe at the treatment site will be killed at an even rate.

In some embodiments, the fiber is single use. In some embodiments, the use of a flexible inner tube allows for the sharp needle to be removed by being withdrawn over the inner tube to allow for longer term use of the fiber.

In use, the introducer and the inner tube are pushed through tissue such that the distal end of the introducer is positioned adjacent to a target tissue. The introducer can be withdrawn from the body, leaving only the inner tube such that the one or more fibers can be delivered through the inner tube to the target tissue. In some embodiments, when longer term delivery of light is needed, the inner tube can be attached to the skin, such as with tape, while the light is still on.

All patents, patent applications, and published references cited herein are hereby incorporated by reference in their entirety. It will be appreciated that several of the above-disclosed and other features and functions, or alternatives thereof, may be desirably combined into many other different systems or applications.

We claim:

1. A device, comprising:
a delivery catheter having an elongated shaft and an inner lumen therethrough, the delivery catheter being configured to pass through tissue such that a distal end of the delivery catheter is positioned at a target tissue;
a support in the form of an inner slidable tube that is configured to be positioned inside the inner lumen of the delivery catheter, the support being configured to provide rigid or semi-rigid support for the delivery catheter during insertion of the delivery catheter through tissue; and
one or more optical fibers configured to pass through the inner slidable tube and being configured to deliver light energy to provide an antimicrobial effect to the target tissue,
wherein the delivery catheter is configured to be movable between a first position in which the one or more optical fibers are positioned within the delivery catheter and a second position in which at least a distal potion of the one or more optical fibers are configured to extend past the distal end of the delivery catheter.

2. The device of claim 1, wherein a distal end of the delivery catheter comprises a deflector component to divert a distal end at least one of the one or more optical fibers as it is being advanced from a distal end of the inner tube.

3. The device of claim 2, wherein the deflector component comprises a cut out portion and a distal ramp can that is configured to act as a deflector of the one or more optical fibers.

4. The device of claim 1, wherein the delivery catheter includes a proximal end with a head and a distal end having an angled tip.

5. The device of claim 1, wherein at least one of the delivery catheter or the inner tube includes one or more optical windows to allow light from the one or more optical fibers to escape from the delivery catheter or the inner tube.

6. The device of claim 5, wherein locations and number of the one or more optical windows is based on the number of the one or more optical fibers or treatment required at the target tissue.

7. The device of claim 1, wherein the one or more optical fibers are configured to disperse the light energy evenly over a length of the one or more optical fibers in both longitudinal and circumferential directions.

8. The device of claim 7, wherein the one or more optical fibers include a cladding covering an outer surface thereof, and wherein at least a portion of the cladding of the one or more optical fibers is removed from an outer surface of the one or more optical fibers to achieve the even dispersion of the light energy.

9. The device of claim 1, wherein the antimicrobial effect of the light energy is configured to kill bacteria to treat infections.

10. The device of claim 1, wherein the light energy has illumination wavelengths from about 400 nm to about 475 nm.

11. The device of claim 1, wherein the light energy has illumination wavelengths from about 380 nm to about 500 nm.

12. The device of claim 1, wherein the light energy has illumination wavelengths from about 405 nm to about 470 nm.

13. A device, comprising:
a delivery catheter having an elongated shaft and an inner lumen therethrough, the delivery catheter being configured to pass through tissue such that a distal end of the delivery catheter is positioned at a target tissue, the delivery catheter including a proximal head;
a support in the form of an inner slidable tube that is configured to be positioned inside the inner lumen of the delivery catheter, the support being configured to provide rigid or semi-rigid support for the delivery catheter during insertion of the delivery catheter through tissue; and
one or more optical fibers configured to pass through the inner slidable tube and being configured to deliver light energy to provide an antimicrobial effect to the target tissue,
wherein at least one of the delivery catheter or the inner tube includes one or more optical windows to allow light from the one or more optical fibers to escape from the delivery catheter or the inner tube.

14. The device of claim 13, wherein a distal end of the delivery catheter comprises a deflector component to divert a distal end at least one of the one or more optical fibers as it is being advanced from a distal end of the inner tube.

15. The device of claim 14, wherein the deflector component comprises a cut out portion and a distal ramp can that is configured to act as a deflector of the one or more optical fibers.

16. A method for treating tissue, comprising:
delivering a delivery catheter to a tissue such that a distal end of the catheter is positioned at a target tissue;
delivering one or more optical fibers through the delivery catheter to the tissue;
moving the delivery catheter between a first position in which the one or more optical fibers are positioned within the delivery catheter and a second position in which at least a distal potion of the one or more optical fibers extend past the distal end of the delivery catheter;
activating a light source engaging the one or more optical fibers; and
delivering light energy from the light source to the one or more optical fibers to provide an antimicrobial effect to the tissue, the one or more optical fibers dispersing the light energy evenly over a length of the one or more optical fibers in both longitudinal and circumferential directions.

17. The method of claim 16, wherein the antimicrobial effect of the light energy is configured to kill bacteria to treat infections.

18. The method of claim 16, wherein the light energy has illumination wavelengths from about 400 nm to about 475 nm.

19. The method of claim 16, wherein the light energy has illumination wavelengths from about 380 nm to about 500 nm.

20. The method of claim 16, wherein the light energy has illumination wavelengths from about 405 nm to about 470 nm.
